(19) United States
(12) Patent Application Publication
(10) Pub. No.: US 2008/0044384 A1
(43) Pub. Date: Feb. 21, 2008

(54) Recombinant Human Cytomegalovirus And Vaccines Comprising Heterologous Antigens

(75) Inventors: George Kemble, Saratoga, CA (US); Harry Greenberg, Palo Alto, CA (US); Gregory Duke, Redwood City, CA (US)

Correspondence Address: JOHNATHAN KLEIN-EVANS, ONE MEDIMMUNE WAY, GAITHERSBURG, MD, 20878, US

(73) Assignee: Medlmmune Vaccines, Inc., Gaithersburg, MD (US)

(21) Appl. No.: 11/571,081

(22) PCT Filed: Jun. 24, 2005
(86) PCT No.: PCT/US05/22734

§ 371 Date: Aug. 1, 2007

Related U.S. Application Data
(60) Provisional application No. 60/583,188, filed on Jun. 25, 2004.

(51) Int. Cl. A61K 35/76; A61P 43/00; C12N 7/01; C12N 7/04

(52) U.S. Cl. 424/93.2; 435/235.1; 435/236

(57) ABSTRACT

The present invention relates to recombinant HCMV (human cytomegalovirus) expressing a pp65 polypeptide or fragment thereof fused to a heterologous or non-native polypeptide, in particular immunogenic and/or antigenic polypeptides. In particular, the heterologous gene products include antigenic or immunogenic polypeptides from a variety of pathogens, cellular genes, tumor antigens, and viruses. The recombinant viruses may advantageously be used in vaccine formulations including vaccines against a broad range of pathogens and antigens.

### FIELD OF THE INVENTION

The present invention relates to recombinant human cytomegaloviruses (HCMV) expressing a pp65 polypeptide or fragment thereof fused to a heterologous polypeptide. In particular, the present invention encompasses immunogenic preparations (e.g., vaccines) comprising recombinant HCMV expressing a pp65-heterologous polypeptide fusion, wherein the heterologous polypeptide of the invention is preferably an antigenic and/or immunogenic polypeptide. The heterologous polypeptide may be any polypeptide useful to elicit an immune response when administered to an animal, e.g., a polypeptide derived from a pathogenic microorganism or associated with a tumoral disorder (e.g., a tumor-associated antigen). In one embodiment, the recombinant HCMV of the invention expresses more then one heterologous polypeptide.

In another embodiment of the invention, the heterologous polypeptide of the invention is an antigenic or immunogenic polypeptide from a virus, e.g. an RNA virus, including but not limited to human immunodeficiency virus (HIV) and hepatitis C virus (HCV).

In still another embodiment, the heterologous polypeptide of the invention is an antigenic or immunogenic polypeptide from a pathogen, including but not limited to, M. tuberculosis and Leishmania. In particular, the heterologous polypeptide is preferably derived from an intracellular pathogen.

In yet another embodiment, the heterologous polypeptide of the invention is a tumor-associated antigen.

Further, in a particular embodiment, the HCMV of the invention may be a live, attenuated or inactivated (e.g., replication deficient) virus or a recombinant virus selected from, but not limited to, the following HCMV strains: AD169, Toledo and Towne. The HCMV of the invention may also be a chimeric virus comprising polynucleotide sequences derived from, but not limited to, AD169 and/or Towne and/or Toledo strains of HCMV.

The present invention also includes a recombinant HCMV expressing a heterologous polynucleotide operatively linked to a CMV pp65 promoter. The heterologous polynucleotide preferably encodes for a polypeptide useful to induce a protective immune response. In one embodiment the encoded polypeptide is derived from a virus or an intracellular pathogen. In still another embodiment the encoded polypeptide is derived from a tumor-associated antigen.

### BACKGROUND OF THE INVENTION

All articles, patents and other materials referred to below are specifically incorporated herein by reference.

The development of cell-mediated immunity, particularly that involving a CD8+ T-cell mediated Cytotoxic T Lymphocyte (CTL) response represents an essential host factor in the control of persistent infection and other disease states. CTL responses are known to play an important role in the clearance of viral infection (R. A. Good, 1991, Immunol. Today 12, 233-236, also reviewed in: Field's Virology, 2001, (4^{th} ed.), Editors Knipe D M and Howley P M, Lippincott Williams & Wilkins, Philadelphia) and also the development of beneficial anti-tumor responses (A. Anichini et al., 1987, Immunol. Today 8, 385-389). Because CTL responses are difficult to induce in the absence of an active infection, current vaccine strategies for the prevention or treatment of numerous infections and diseases are of limited efficacy.

Vaccines have traditionally been used as a means to protect against disease caused by infectious agents. However, with the advancement of vaccine technology, vaccines have been used in additional applications that include, but are not limited to, control of mammalian fertility, modulation of hormone action, and prevention or treatment of tumors. The primary purpose of vaccines used to protect against a disease is to induce immunological memory to a particular microorganism. More generally, vaccines are needed to induce an immune response to specific antigens, whether they belong to a microorganism or are expressed by tumor cells or other diseased or abnormal cells. Division and differentiation of B- and T-lymphocytes that have surface receptors specific for the antigen generate both specificity and memory.

In order for a vaccine to induce a protective immune response, it must fulfill the following requirements: 1) it must include the specific antigen(s) or fragment(s) thereof that will be the target of protective immunity following vaccination; 2) it must present such antigens in a form that can be recognized by the immune system; and 3) it must activate antigen presenting cells (APCs) to present the antigen to CD4 + T-cells, which in turn induce B-cell differentiation and the CD8+ mediated CTL response.

Conventional vaccines contain suspensions of attenuated or killed microorganisms, such as viruses or bacteria, incapable of inducing severe infection by themselves, but capable of inducing a protective immune response to the unmodified (or virulent) species when inoculated into a host. Usage of the term “vaccine” has now been extended to include essentially any preparation intended for active immunologic prophylaxis (e.g., preparations of killed microbes of virulent strains or living microbes of attenuated (variant or mutant) strains; microbial, fungal, plant, protozoan, or metazoan derivatives or products; synthetic vaccines). Examples of vaccines include, but are not limited to, cowpox virus for inoculating against smallpox, tetanus toxoid to prevent tetanus, whole-inactivated bacteria to prevent whooping cough (pertussis), polysaccharide subunits to prevent streptococcal pneumonia, recombinant proteins to prevent hepatitis B and monoclonal antibodies to prevent respiratory syncytial virus.

Conventional vaccination with inactivated or attenuated organisms or their products has been shown to be an effective method for increasing host resistance and ultimately has led to the eradication of certain common and serious infectious diseases. However, their use has been limited because their efficacy generally requires specific, detailed knowledge of the molecular determinants of virulence. Moreover, the use of attenuated pathogens in vaccines is associated with a variety of risk factors that, in most cases, prevent their safe use in humans.

Antigens may also be delivered in currently accessible recombinant vaccine vectors based on poxviruses and adenoviruses but despite promise, these vectors have several major limitations that are impediments to development. These limitations include the fact that the immunity they induce wanes rapidly and that they are ineffective in individuals with preexisting immunity to the vector.

In addition, the use of synthetic vaccines, such as purified proteins or other molecules, is limited because they are often non-immunogenic or non-protective. The use of available adjuvants to increase the immunogenicity of synthetic vaccines is often not an option because of unacceptable side effects induced by the adjuvants themselves.

Thus, a safe adaptable human virus vaccine vector that is capable of infecting and inducing sustained, high level cellular immunity, preferably the CTL response, in both naïve and immune individuals without undesired side effects would provide a practical solution to these problems, particularly if the vaccine vector itself were to bring a benefit to individuals.

Human cytomegalovirus (HCMV) belongs to the herpes virus family. Several proteins encoded by CMV are known to be recognized by the cellular immune system and elicit the CTL response. Borysiewicz et al., (1988, J. Exp. Med. 168: 919) described the role of specific HCMV proteins in CTL induction. The non-virion, immediate early (IE) proteins of HCMV, as well as the envelope glycoprotein, gB, activate CTL function, however, the internal matrix proteins of the virus, pp65 and pp150, are more prevalent immune targets (Gyulai et al., 2002, J. Infect. Dis. 181: 1537-46; McLaughlin-Taylor et al., 1994, J. Med. Virol. 43: 103). pp65 is the immunodominant protein: 70-90% of all HCMV-specific CTL recognize this protein (Gyulai et al., supra; Wills et al., 1996, J. Virol. 70: 7569). Approaches using these CMV encoded proteins, pp65 in particular, to develop vaccines against HCMV have been published by various authors (CMV vaccines are reviewed in Gonczol et al., 2001, Expert Opin. Biol. Ther. 1: 401, Plotkin, 2001, Arch. Virol. Suppl. 121-34).

Bruggeman et al., in U.S. Pat. No. 5,800,981 (issued Sep. 1, 1998) describes a recombinant protein termed a “combined antigen” having at least three HCMV epitopes for use in assays for the detection of HCMV-specific antibodies. They indicate that this “combined antigen” could be used as a vaccine to confer protective immunity against HCMV-medicated diseases, although no details are provided.

Pande et al., in U.S. Pat. No. 6,207,161 (issued Mar. 27, 2001) disclose a method using purified pp65 protein or polypeptides to stimulate a HCMV-specific CTL response in isolated HCMV seropositive, but not seronegative, blood mononuclear leukocytes. They hypothesize that pp65 is an appropriate antigen to target in efforts to provide hosts with immunity to CMV however, no details are provided.

Prieur et al., in U.S. Pat. No. 6,605,467 (issued Aug. 12, 2003) disclose that a fusion protein comprised of part of pp65 and part of IE1 can activate IE1-specific CD4+ and pp65-specific CD8+ T-cells in vitro. They assert that the fusion proteins and the corresponding nucleotide sequences can be used for the preparation of a synthetic vaccine for preventing HCMV infections.

Zaia et al., in US Patent Publication 2003/0165522 (published Sep. 4, 2003) disclose the production, purification and characterization of a point mutant, kinase inactive, pp65 protein. They further disclose that a nucleotide sequence corresponding to the kinase inactive pp65 protein could elicit a CTL response in transgenic HLA-A2.1 mice.

Diamond in US Patent Publication 2003/0190328 (published Oct. 9, 2003) discloses the sequence of specific pp65 peptides that should elicit an HLA allele-specific CTL response. They further disclose that one of the peptides, peptide 495, does elicit a HLA-A2 subtype-specific CTL response. They hypothesize that specific combinations of pp65 peptides could be used to formulate a synthetic vaccine useful to vaccinate a multi-ethnic human population against HCMV but no data are provided.

Plachter in U.S. Pat. No. 6,713,070 (issued Mar. 30, 2004) disclose the hypothesis that HCMV “dense bodies”, described as defective viral particles produced during the infection of primary human fibroblasts, could be used as a vaccine against HCMV. However, only limited mouse data are disclosed. They further hypothesize that recombinant “dense bodies” in which a heterologous polypeptide is fused to pp65 or ppUL123 could be used as a vaccine against other pathogens. No data is provided either for the production of such recombinant dense bodies or their use in any model.

All of the vaccine studies described above rely on synthetic formulations or recombinant vaccine strategies that suffer from the limitations described above. In addition, these vaccine strategies have predominantly focused on the formulation of HCMV derived proteins and/or peptides into a vaccine useful in conferring protective immunity specifically to HCMV, they do not however, address the underlying medical need for a more generally useful and adaptable human vaccine vector. In particular, they do not address the need for a vaccine formulation that can elicit a robust and long lived CTL response and thus confer immunity to a broad range of other antigens.

Immunity to HCMV differs in both magnitude and quality from immunity to other viral pathogens in important ways that indicate that this virus should be harnessed as a vector to the potential benefit of vaccination. Three unique biological features of HCMV infection and immunity are central to this rationale: 1) the ability of this virus to induce remarkably broad and intense CD4+ and CD8+ responses, 2) the observation that the proportion of T cells that retain responsiveness to viral antigens remains very high throughout life and 3) the ability of HCMV to re-infect, replicate and boost immunity in the face of an existing immune response (reviewed in: Cytomegalovirus Biology and Infection, 1991 (2^{nd} ed.) Editor Ho, M., Plenum Med. Press, New York). In addition, the large size, manipulability, well characterized, strong gene expression system and ready growth of HCMV in culture all provide a solid foundation to utilize this virus as a vaccine vector.

Infection with HCMV occurs frequently, as evidenced by the high percentage (over 50%) of adults having antibodies to this virus. The proportion of seropositive persons in a population depends on several factors including socioeconomic status and geography; some populations have seropositive rates exceeding 90% of the population prior to adolescence. Like other herpes viruses, HCMV can establish life-long latency after initial infection (Stevens, 1989, Microbiol. Rev. 53: 318-332; Bruggeman, 1993, Virchows Arch, B cell Pathol. 64: 325-333). It is believed that this latency is essential for the long lived immune response elicited by HCMV. Infection in the normal immunocompetent individual is mild or asymptomatic and infection with HCMV has a reputation for being very benign. Thus, a HCMV-based vector does not need significant attenuation to exhibit safety in much of the world as infection is already occurring without apparent disease association in young children; however additional safety characteristics could be introduced by adapting existing mutant strains or engineering specific attenuation characteristics that would reduce cell tropism or ability to replicate in some or all target cells in the host. These would provide additional measures of safety.

Spaete et al., in 1987, Proc. Natl. Acad. Sci. USA 84: 7213, Mocarski et al., in European Patent application EP0277773A1 (published Aug. 10, 1988) and Hock et al. in U.S. Pat. No. 5,830,745 (issued Nov. 3, 1998) disclose methods for expressing a heterologous protein in HCMV. All groups suggest the use of such recombinant viruses as vaccines but none present any supporting data. In addition, they did not focus on the creation of a cellular immune response against non-HCMV antigens nor did they consider the lack of an animal model to study the efficacy of such a vaccine. The recombinant HCMV described by both the Spaete et al. and Hock et al. groups would not a priori be expected to stimulate a robust cellular response to the non-HCMV gene product.

Karrer et al. in 2004, J. Virol. 78: 2255 present data using a recombinant mouse CMV (MCMV) in which a heterologous pathogen antigen is fused to the IE2 gene. They purport that the recombinant MCMV can produce a long lasting antigen specific CD8+ response in mice. While they argue that such a response could be protective in the mouse model, limited supporting data were provided. In addition, they speculate that a similar approach could be used in humans, but no data were provided. Yet another shortcoming of the teachings of Karrer et al. is the low level of CD8+ response generated in their mouse model would not be expected to be sufficient for protection.

The present invention addresses the need for a safe, adaptable, recombinant human vaccine vector capable of inducing high titer, long lived T-cell response in the absence of severe illness or adverse effects in both naïve and immune individuals utilizing HCMV as a vaccine vector. In addition, the present invention provides for a novel strategy to evaluate recombinant HCMV vaccine candidates in an animal model.

Citation or discussion of a reference herein shall not be construed as an admission that such is prior art to the present invention.

### SUMMARY OF THE INVENTION

The present invention provides for the first time a recombinant HCMV vaccine vector that is able to stimulate a robust cellular immune response, especially a Cytotoxic T Lymphocyte (CTL) response, to a foreign antigen inserted into the HCMV genome. In a specific embodiment, the present invention provides vaccine compositions that are able to stimulate a CTL response against an intracellular pathogen or viral infection (e.g., M. tuberculosis, Leishmania, and other pathogens HCV, HIV, other viruses). In still another embodiment, the present invention provides vaccine compositions that are able to stimulate a CTL response to a tumor-associated antigen (TAA).

The present invention provides for the first time a recombinant HCMV formulated as an immunogenic preparation (e.g., a vaccine) that is able to induce a high titer, long lived T-cell response to a heterologous polypeptide, in both naïve and immune individuals.

The present invention relates to recombinant HCMV viruses engineered to express a pp65 polypeptide or fragment thereof fused to a heterologous or non-native polypeptide, in particular immunogenic and/or antigenic polypeptides.

In one embodiment, the present invention relates to recombinant Toledo, Towne or AD169 HCMV strains. In another embodiment, the present invention relates to recombinant HCMV that contains modifications that result in a chimeric virus with a phenotype more suitable for use in vaccine formulations. In particular the invention specifically includes chimeras of the AD169, Towne and Toledo-strains of HCMV. The invention also allows for the reconstitution of infectious virus with the desired genetic characteristics from available overlapping cosmids and bacmids.

The recombinant CMV can exist as a naked polynucleotide, but in the methods disclosed herein is generally encapsulated to form an infectious and biologically active virus. Furthermore, the recombinant CMV provided need not include the entire genome. As already indicated, certain genes can be deleted to attenuate the virus. Other genes can also be deleted or modified, provided the resulting virus is still capable of infecting the desired host or replicating and/or disseminating to the degree necessary to elicit an immune response.

The present invention also relates to engineered recombinant HCMV that expresses one, two, three or more heterologous polypeptides, including but not limited to, polypeptide antigens and other products from a variety of pathogens, cellular genes, tumor antigens, and viruses. The heterologous polypeptides may be expressed as part of a fusion protein, for example, fused to the endogenous pp65 protein. Alternatively, the heterologous polypeptides may be expressed independently of any endogenous protein. A heterologous polypeptide expressed as a fusion protein may further incorporate a protein cleavage site to allow for the cleavage of the expressed fusion protein to separate the heterologous polypeptide from the protein to which it was fused.

In one embodiment, the invention relates to engineered recombinant HCMV expressing a polypeptide derived from retroviruses, e.g., HIV or HCV. In a preferred embodiment, the polypeptide is an immunogenic and/or antigenic polypeptide derived from HIV (e.g., a Env, Gag, Pol or Nef) or HCV (e.g., NS3, NS4a, NS4b, or NS5).

The present invention provides for the first time a recombinant HCMV that expresses a heterologous immunogenic or antigenic polypeptide that can elicit a robust immune response. In a specific embodiment, the heterologous polypeptide of the present invention is derived from a virus that can cause respiratory tract infections e.g., PIV, SARS coronavirus, hMPV, and RSV.

In yet another preferred embodiment, the polypeptide is a cancer antigen (e.g., a tumor-associated antigen such as prostate-specific antigen (PSA) or cancer antigen 125 (CA 125). In still another preferred embodiment the polypeptide is derived from a protein known to be specifically up regulated on the surface of tumor cells such as EphA2 or EphA4.

In accordance with the present invention, a recombinant HCMV of the invention expresses the HCMV pp65 polypeptide or fragment thereof fused to one, two, three or more heterologous polypeptides. In addition, the heterologous polypeptides may be a full-length antigenic polypeptide or an immunogenic and/or antigenic fragment thereof. The heterologous polypeptide may further incorporate a protein cleavage site.

The present invention also relates to a recombinant HCMV expressing a heterologous polypeptide operatively linked to a CMV pp65 promoter. It is specifically contemplated that the endogenous pp65 promoter could be used or, alternatively, an additional pp65 promoter, with or without pp65 protein coding sequence, is operably fused directly to a heterologous antigen by standard recombinant techniques.

The present invention provides for the first time the use of a recombinant HCMV to stimulate a robust cellular immune response, especially a cytotoxic T-cell response, to a foreign antigen inserted into the HCMV genome.

In a specific embodiment, the present invention provides a recombinant HCMV formulated as a vaccine that is able to confer protection, in humans or animals, against a variety of pathogens, cellular genes, and viruses.

In another embodiment, the present invention provides for the first time the use of recombinant HCMV expressing a heterologous polypeptide as method for the prevention or treatment of cancer in humans.

### BRIEF DESCRIPTION OF THE FIGURES

FIG. 1 depicts the HCV proteins to be fused to the UL83 gene of HCMV. Inset is the amino acid sequence of a portion of the NS3 protein. HCV_NS3 is the wildtype HCV sequence (SEQ ID NO: 1), HCV_NS3_{—}4A_{—}4Bser- is the serine protease mutant sequence (SEQ ID NO: 2). Arrows indicated mutated HCV_NS3 residues in the serine protease mutant.

FIG. 2 outlines the construction of recombinant HCMV expressing HCV_NS3_{—}4A_{—}4B (ser-) amino acid sequences fused to UL83 (pp65), the presence of the FMDV 2A cleavage signal allows cleavage of the fusion protein into it's two distinct protein components (e.g., a functional pp65 protein and the HCV antigen). Two constructs are depicted. Panel A represents a construct for expression from native UL83 locus by fusing the HCV NS3 sequences to the endogenous UL83 gene. Panel B represents a construct for epitotic expression where a cassette comprising a second UL83 gene fused to HCV NS3 sequences is inserted elsewhere in the genome.

FIG. 3 is a Southern blot analysis of the recombinant HCMV viral construct containing the UL83-NS3_{—}4 fusion. Panel A shows the restriction enzyme digest banding pattern of the both the recombinant virus (Toledo 2A HCV) and the wild type virus (Toledo). Panel B shows the corresponding autoradiograph probed with an HCV NS3_{—}4 specific probe. A band at approximately 9 kb is observed only in the recombinant virus demonstrating the presence of the HCV NS3_{—}4 gene in the HCMV viral backbone.

FIG. 4 is a Western blot analysis of cell extracts 24, 48 and 72 hours post infection (hpi). The blot was probed with anti-pp65 (the UL83 gene product) antibody. The arrow indicates the expected size of the pp65 protein. Expression of pp65 can be detected by 48 hpi in the wild type Toledo parental strain and is strongly expressed by 72 hpi. All of the infected cells including the two independent recombinant Toledo 2A HCV clones show a series of pp65 protein bands which correspond to the reported posttranslationally modified forms and cleavage products.

FIG. 5 is a Western blot analysis of cell extracts 48 and 72 hours post infection (hpi). The blot was probed with anti-HSV NS3 antibody. The arrow indicates the expected size of the HCV NS3_{—}4 protein. Only the recombinant Toledo 2A HCV strain is seen to express the NS3 protein. As expected the time of expression correlates with that of pp65 in the wild type Toledo parental strain (see FIG. 4).

### DETAILED DESCRIPTION

The present invention provides for the first time a recombinant HCMV immunogenic preparation that is able to stimulate a robust cellular immune response, especially a Cytotoxic T Lymphocyte (CTL) response, to a foreign antigen inserted into the HCMV genome. In a specific embodiment, the present invention provides a recombinant HCMV immunogenic preparation that is able to stimulate a CTL response against a viral infection or intracellular pathogen. In still another embodiment, the present invention provides an immunogenic preparation that is able to stimulate a CTL response to a tumor-associated antigen (TAA). The present invention provides for the first time a recombinant HCMV formulated as a vaccine that is able to induce a high titer long lived T-cell response, to a heterologous polypeptide, in both naïve and immune individuals.

The present invention relates to recombinant HCMV and DNA that may be engineered to express pp65 polypeptide or fragment thereof fused to a heterologous or non-native polypeptide, in particular, to express fusions of pp65 and immunogenic and/or antigenic polypeptides and peptides. In one embodiment, the recombinant HCMV is engineered to express sequences that are non-native to the HCMV genome. The HCMV that is modified can be a wild-type strain, a clinical strain, an attenuated strain, and/or a genetically engineered strain. The present invention also relates to recombinant HCMV that contains certain modifications that result in chimeric viruses with phenotypes more suitable for use in immunogenic preparations (e.g., vaccines).

In accordance with the present invention, a chimeric virus of the invention is a recombinant HCMV that further comprises one or more heterologous nucleotide sequences. In accordance with the invention, a non-native sequence is one that is different from the native or endogenous genomic sequence due to one or more mutations, including, but not limited to, point mutations, rearrangements, insertions, deletions, etc. to the genomic sequence that may or may not result in a phenotypic change. These recombinant and chimeric viruses may be used to prepare immunogenic preparations (e.g., vaccines) suitable for administration to humans or animals. For example, the chimeric viruses of the invention may be used in vaccine formulations to confer protection against intracellular pathogens or viral infections or to treat a tumor related disorder.

In one embodiment, the present invention relates to recombinant HCMV constructs that are engineered to express one, two, three or more heterologous polypeptides, preferably foreign antigens and/or antigenic fragments from a variety of pathogens, cellular genes, tumor antigens, and viruses.

It will be appreciated that it is not necessary (or always desirable) to express the entire polypeptide encoded by a pathogen, cellular gene, tumor related protein or virus, and typically, one or more an immunogenic fragments of the polypeptides are expressed. When fragments are used, suitable immunogenic sequences are known or can be determined using routine art-known methods. See, e.g., Ausubel, F. M., et al., 1998, Current Protocols in Molecular Biology, John Wiley & Sons, chapter 11.15. Typically, the expressed immunogenic polypeptide is at least 6 amino acids in length, more often at least about 8, and sometimes at least about 10, at least about 20, at least about 50 residues, or even full-length.

In accordance with the present invention, a recombinant virus is one derived from a human cytomegalovirus (HCMV) that is encoded by endogenous and/or native genomic sequences and/or non-native genomic sequences. In accordance with the invention, a non-native sequence is one that is different from the native or endogenous genomic sequence due to one or more mutations, including, but not limited to, point mutations, rearrangements, insertions, deletions, etc. to the genomic sequence that may or may not result in a phenotypic change. In particular, the invention relates to recombinant CMV AD169 (ATCC #VR 538), human CMV Towne (ATCC #VR 977), human CMV Davis (ATCC #VR 807), human CMV Toledo (Quinnan et al, 1984, Arm Intern Med 101: 478-83), and others as well as DNA molecules coding for the same. “ATCC” is the American Type Culture Collection, 10801 University Boulevard, Manassas, Va. 20110-2209 USA. The 230-kb dsDNA genome of human and murine CMV were sequenced (see, e.g., Chee et al., 1990, Curr. Top. Microbiol. Immunol. 154: 125-169; also see Rawlinson, 1996, J. Virol. 70: 8833-49, both incorporated herein in their entirety). The invention also allows for the reconstitution of infectious virus with the desired genetic characteristics from available overlapping cosmids and bacmids (see, e.g., U.S. Pat. Nos. 6,277,621 and 6,692,954, both incorporated herein in their entirety). The present invention also relates to recombinant HCMV that contains modifications that result in chimeric viruses with phenotypes more suitable for use in vaccine formulations (see, e.g., U.S. Pat. Nos. 6,291,236, 6,646,170, 5,925,751 and 6,040,170, incorporated herein in their entirety).

In accordance with the present invention, a chimeric virus of the invention is a recombinant HCMV that further comprises one or more heterologous nucleotide sequences. In accordance with the invention, a chimeric virus may be encoded by a nucleotide sequence in which heterologous nucleotide sequences have been added to the genome or in which nucleotide sequences have been replaced with heterologous nucleotide sequences.

The present invention also relates to engineered recombinant HCMV that encodes combinations of heterologous sequences which encode gene products, including but not limited to, genes from different strains of HCMV, other viruses, pathogens, cellular genes, tumor antigens, or combinations thereof.

The present invention also provides vaccine and immunogenic preparations comprising chimeric HCMV expressing one or more heterologous antigenic sequences. In a specific embodiment, the present invention provides multivalent immunogenic compositions, including bivalent and trivalent compositions. The multivalent immunogenic compositions of the invention may be administered in the form of one recombinant HCMV expressing each heterologous antigenic sequence or two or more recombinant HCMV each encoding different heterologous antigenic sequences. In one embodiment, the immunogenic preparation of the invention comprises chimeric recombinant HCMV expressing one, two or three heterologous polypeptides, wherein the heterologous polypeptides can be encoded by polynucleotide sequences derived from different viral, pathogen, cellular or tumor antigens or combinations thereof. In another embodiment, the immunogenic preparation of the invention comprises chimeric recombinant HCMV expressing one, two or three similar antigens derived from different strains of viruses or other pathogens.

In certain embodiments, the invention provides a vaccine formulation comprising the recombinant or chimeric virus of the invention and a pharmaceutically acceptable excipient. In specific embodiments, the vaccine formulation of the invention is used to modulate the immune response of a subject, such as a human, a primate, a horse, a cow, a sheep, a pig, a goat, a dog, a cat, a rodent or a subject of avian species. In a more specific embodiment, the vaccine is used to modulate the immune response of a human. In another embodiment, the present invention relates to vaccine formulations for veterinary uses wherein the recombinant CMV would be derived from the appropriate species. The vaccine preparation of the invention can be administered alone or in combination with other vaccines or other prophylactic or therapeutic agents.

1. Human Immunodeficiency Virus (HIV)

HIV infection and disease remains uncontrolled in much of the world. An effective vaccine would have the potential to prevent infection and/or disease in naïve individuals and impede progression to AIDS in those already infected. HIV antigens are known; their worldwide geographic distribution has been established. However candidate subunit glycoprotein vaccines that elicited a strong humoral response have been tried and failed. Despite the existence of effective vaccines for many pathogens and despite a growing level of scientific understanding on how to develop vaccines, the path to a successful HIV vaccine remains elusive. Both epidemiology studies and immune-depletion studies in animals have demonstrated the importance of cellular immunity in preventing disease progression with HIV. The live, DNA virus-vectored HCMV vaccine approach described here can induce sustained cellular immunity and should provide exceptional benefit in combating HIV/AIDS.

It is specifically contemplated that the present invention, an engineered recombinant HCMV virus, contains at least one polynucleotide sequence derived from HIV.

In a preferred embodiment, the polynucleotide sequence encodes an antigenic polypeptide of an HIV isolate (e.g., HXB2, LAV-1, NY5, BRU, SF2), which causes diseases in animals (preferably humans) such as AIDS.

The genomic sequence of HIV as exemplified by GenBank Accession Nos. K03455 and AF033819 which are explicitly provided herein to provide examples of polynucleotides (including open reading frames, fragments, variants, or complements thereof), which may be engineered into the viral vectors of the invention.

Some representative examples of polypeptide antigens of HIV include but are not limited to, Gag, Pol, Vif and Nef (Vogt et al., 1995, Vaccine 13: 202-208); HIV antigens gp120 and gp160 (Achour et al., 1995, Cell. Mol. Biol. 41: 395-400; Hone et al., 1994, Dev. Biol. Stand. 82: 159-162); gp41 epitope of human immunodeficiency virus (Eckhart et al., 1996, J. Gen. Virol. 77: 2001-2008) derived from an HIV isolate selected from the group including but not limited to: HXB2, LAV-1, NY5, BRU, SF2. These references list preferred polypeptide antigens of the invention and are incorporated by reference herein.

In another preferred embodiment, the engineered recombinant HCMV comprises a polynucleotide sequence which is at least 60% identical, or at least 70% identical, or at least 75% identical, or at least 80% identical, or at least 85% identical, or at least 90% identical, or at least 95% identical, or at least 99% identical to a polynucleotide sequence encoding an antigenic polypeptide. In addition, the polynucleotide sequence may encode a full-length antigenic polypeptide or an immunogenic fragment thereof.

Furthermore, the invention relates to engineered recombinant HCMV that contains nucleotide sequences derived from more then one preferred HIV antigen. The invention also encompasses recombinant HCMV that are engineered to encode antigens from different isolates and strains of HIV.

2. Hepatitis C Virus (HCV)

Antigenic heterogeneity of different strains of Hepatitis C Virus (HCV) is a major problem in development of effective vaccines against HCV. Antibodies or CTLs specific for one strain of HCV typically do not protects against other strains. Multivalent vaccine antigens that simultaneously protect against several strains of HCV would be of major importance when developing efficient vaccines against HCV. The HCV envelope genes, which encode envelope proteins E1 and E2, have been shown to induce both antibody and lymphoproliferative responses against these antigens. The hypervariable region I (HVR1) of the envelope protein E2 of HCV is the most variable antigenic fragment in the whole viral genome and is primarily responsible for the large inter- and intra-individual heterogeneity of the infecting virus (Puntoriero et al. (1998) EMBO J. 17: 3521-33). The recombinant HCMV of the invention can be readily engineer to express multiple HCV antigens and thus could be utilized to elicit long lived cellular protection again multiple HCV strains.

It is specifically contemplated that the present invention, an engineered recombinant HCMV virus, contains at least one polynucleotide sequence derived from HCV.

In a preferred embodiment, the polynucleotide sequence encodes an antigenic polypeptide of an HCV isolate (e.g., genotype 1a, 1b, 2a, 2b and 3a-11a), which causes diseases in animals (preferably humans) such as hepatitis C.

In another preferred embodiment, the engineered recombinant HCMV comprises one or more polynucleotide sequences which are at least 60% identical, or at least 70% identical, or at least 75% identical, or at least 80% identical, or at least 85% identical, or at least 90% identical, or at least 95% identical, or at least 99% identical to a polynucleotide sequence encoding an antigenic polypeptide (e.g., nucleocapsid protein in a secreted or a nonsecreted form, core protein (pC); E1 (pE1), E2 (pE2) (Saito et al., 1997, Gastroenterology 112: 1321-1330), NS3, NS4a, NS4b and NS5 (Chen et al., 1992, Virology 188: 102-113), derived from an HCV isolate selected from the group including but not limited to: genotypes 1a, 1b, 2a, 2b and 3a-11a. In addition, the polynucleotide sequence may encode a full-length antigenic polypeptide or an immunogenic fragment thereof.

3. Viruses that Cause Respiratory Tract Illness: RSV, PIV, SARS Coronavirus, and hMPV

Human respiratory syncytial virus (hRSV), a species of the Pneumovirus genus, is the single most important cause of lower respiratory tract infections during infancy and early childhood worldwide (Domachowske, & Rosenberg, 1999, Clin. Microbio. Rev. 12: 298-309). While a vaccine might prevent RSV infection, and/or RSV-related disease, no vaccine is yet licensed for this indication. A major obstacle to vaccine development is safety. A formalin-inactivated vaccine, though immunogenic, unexpectedly caused a higher and more severe incidence of lower respiratory tract disease due to RSV in immunized infants than in infants immunized with a similarly prepared trivalent parainfluenza vaccine (Kim et al., 1969, Am. J. Epidemiol. 89: 422-34; and Kapikian et al., 1969, Am. J. Epidemiol. 89: 405-21).

Parainfluenza viral infection results in serious respiratory tract disease in infants and children. (Tao et al., 1999, Vaccine 17: 1100-8). Infectious parainfluenza viral infections account for approximately 20% of all hospitalizations of pediatric patients that suffer from respiratory tract infections worldwide. An effective antiviral therapy is not available to treat PIV related diseases, and a vaccine to prevent PIV infection has not yet been approved.

Recently, an outbreak of atypical pneumonia, referred to as severe acute respiratory syndrome (SARS) (first identified in Guandon, Province, China), has spread to several countries. The severity of this disease is underscored by a mortality rate of approximately 3 to 6% (See, Marra et al., Science (May 2003)). The cause of this atypical pneumonia has been identified to be a coronavirus. Several isolates have been sequenced and deposited into GenBank. See, e.g., isolates BJO1: GenBank No. AY278488, TOR2: GenBank No. NC_{—}004718, CUHK-W1: GenBank No. AY278554, and HKU-39849: GenBank No. AY278491.

Recently, a new member of the Paramyxoviridae family has been isolated from 28 children with clinical symptoms reminiscent of those caused by hRSV infection, ranging from mild upper respiratory tract disease to severe bronchiolitis and pneumonia (Van Den Hoogen et al., 2001, Nature Medicine 7: 719-24). The new virus was named human 6 metapneumovirus (hWV) based on sequence homology and gene constellation. The study further showed that by the age of five years virtually all children in the Netherlands have been exposed to HMPV and that the virus has been circulating in humans for at least half a century.

The recombinant HCMV of the invention can be harnessed as an effective therapeutic for the prevention of severe respiratory tract disease caused by any one of the agents described above.

It is specifically contemplated that the present invention, an engineered recombinant HCMV virus, contains at least one polynucleotide sequence derived from a virus that can cause severe respiratory tract illness in animals (preferably humans) such as pneumonia or SARS.

In a preferred embodiment, the engineered recombinant HCMV comprises one or more polynucleotide sequences which are at least 60% identical, or at least 70% identical, or at least 75% identical, or at least 80% identical, or at least 85% identical, or at least 90% identical, or at least 95% identical, or at least 99% identical to a polynucleotide sequence encoding an antigenic polypeptide. Antigenic peptides of RSV, HMPV and PIV detailed in: Young et al., in Patent publication WO04010935A2 the teachings of which is incorporated herein by reference in its entirety. Antigenic peptides of SARS corona virus include but are not limited to, the S (spike) glycoprotein, small envelope protein E (the E protein), the membrane glycoprotein M (the M protein), the hemagglutinin esterase protein (the HE protein), and the nucleocapsid protein (the N-protein) See, e.g., Marra et al., “The Genome Sequence of the SARS-Associated Coronavirus,” Science Express, May 2003; BCCA Genome Sciences Centre, GenBank Accession no. NC_{—}004718 (May 2003); GenBank Accession Nos. AY278554, AY278491, and AY278488. These references list preferred polypeptide antigens of the invention and are incorporated by reference herein.

4. Mycobacterium

Tuberculosis is an ancient bacterial disease caused by Mycobacterium tuberculosis that continues to be an important public health problem worldwide and calls are being made for an improved effort in eradication (Morb. Mortal Wkly Rep (Aug. 21, 1998; 47(RR-13): 1-6; Sudre et al., 1992, Bull. World Health Organ. 70: 149-59). It infects over 50 million people and over 3 million people will die from tuberculosis this year. The currently available vaccine, Bacille Calmette-Guerin (BCG) is found to be less effective in developing countries and an increasing number of multidrug-resistant (MDR) strains are being isolated (Sudre et al., 1998, Int. J. Tuberc. Lung Dis. 8: 609-11). A number of specific antigens have been identified; these include the major immunodominant antigen of M. tuberculosis, the 30-35 kDa (a.k.a. antigen 85, alpha-antigen) that is normally a lipoglycoprotein on the cell surface, a 65-kDa heat shock protein, and a 36-kDa proline-rich antigen (Tascon et al. (1996) Nat. Med. 2: 888-92). Thus, by incorporating one or more M. tuberculosis antigen the recombinant HCMV of this invention could serve as a useful tool in the fight to prevent and possibly cure tuberculosis.

It is specifically contemplated that the present invention, an engineered recombinant HCMV virus, contains at least one polynucleotide sequence derived from M. tuberculosis.

In a preferred embodiment, the polynucleotide sequence encodes an antigenic polypeptide of M. tuberculosis, which causes diseases in animals (preferably humans) such as tuberculosis.

In a preferred embodiment, the engineered recombinant HCMV comprises one or more polynucleotide sequences which are at least 60% identical, or at least 70% identical, or at least 75% identical, or at least 80% identical, or at least 85% identical, or at least 90% identical, or at least 95% identical, or at least 99% identical to a polynucleotide sequence encoding an antigenic polypeptide (e.g. Ag85A, Ag85b (Huygen et al., 1996, Nat. Med. 2: 893-898), 65-kDa heat shock protein, hsp65 (Tascon et al., 1996, Nat. Med. 2: 888-892), MPB/MPT51 (Miki et al, 2004, Infect. Immun. 72: 2014-21), MTSP11, MTSP17 (Lim et al., 2004, FEMS Microbiol. Lett. 232: 51-9 and supra) derived from M. tuberculosis. In addition, the polynucleotide sequence may encode a full-length antigenic polypeptide or an immunogenic fragment thereof.

5. Leishmania

Leishmania organisms are intracellular protozoan parasites of macrophages that cause a wide range of clinical diseases in humans and domestic animals. In some infections, the parasite may lie dormant for many years. In other cases, the host may develop one of a variety of forms of leishmaniasis. Leishmaniasis is a serious problem in much of the world, including Brazil, China, East Africa, India and areas of the Middle East. The disease is also endemic in the Mediterranean region, including southern France, Italy, Greece, Spain, Portugal and North Africa. The number of cases of leishmaniasis has increased dramatically in the last 20 years, and millions of cases of this disease now exist worldwide. About 2 million new cases are diagnosed each year, 25% of which are visceral leishmaniasis. There are, however, no vaccines or effective treatments currently available. The recombinant HCMV vaccine approach described here can induce sustained cellular immunity, specifically a robust CTL response that should provide exceptionally useful for the treatment and prevention of leishmaniasis. The recombinant HCMV can readily be adapted to target a large number of different Leishmania organisms and thus can be used throughout the world to combat the multiple causes of leishmaniasis.

It is specifically contemplated that the present invention, an engineered recombinant HCMV virus, contains at least one polynucleotide sequence derived from Leishmania.

In a preferred embodiment, the polynucleotide sequence encodes an antigenic polypeptide of Leishmania, which causes diseases in animals (preferably humans) such as leishmaniasis.

In another preferred embodiment, the engineered recombinant HCMV comprises one or more polynucleotide sequences which are at least 60% identical, or at least 70% identical, or at least 75% identical, or at least 80% identical, or at least 85% identical, or at least 90% identical, or at least 95% identical, or at least 99% identical to a polynucleotide sequence encoding an antigenic polypeptide (e.g. LPG, gp63 (Xu and Liew, 1994, Vaccine 12: 1534-1536; Xu and Liew, 1995, Immunology 84: 173-176), P-2 (Nylen et al, 2004, Scand. J. Immunol. 59: 294-304), P-4 (Kar et al. 2000, J Biol. Chem. 275: 37789-97), LACK (Kelly et al., 2003, J Exp. Med. 198: 1689-98)) derived from Leishmania. In addition, the polynucleotide sequence may encode a full-length antigenic polypeptide or an immunogenic fragment thereof.

6. Cancer Antigens

Immunotherapy has great promise for the treatment of cancer and prevention of metastasis. By inducing an immune response against cancerous cells, the body's immune system can be enlisted to reduce or eliminate cancer (Lee et al., 1999, Nat. Med. 5: 677-85). The highly specific, long lived and robust CTL response obtained using the recombinant HCMV of the invention provides cancer immunotherapies of increased effectiveness compared to those that are presently available. Prevention of metastasis is also a goal in design of cancer vaccines.

It is specifically contemplated that the present invention, an engineered recombinant HCMV virus, contains at least one polynucleotide sequence derived from cancer or tumor associated antigen. The resulting recombinant viruses can be used to generate an immune response against the tumor cells leading to tumor regression in vivo. These vaccines may be used in combination with other therapeutic regimens, including but not limited to, chemotherapy, radiation therapy, surgery, bone marrow transplantation, etc. for the treatment of tumors.

Specific examples of cancers that can be prevented, managed, treated or ameliorated in accordance with the invention include, but are not limited to, cancer of the head, neck, eye, mouth, throat, esophagus, chest, bone, lung, colon, rectum, bladder or other gastrointestinal tract organs, stomach, spleen, skeletal muscle, subcutaneous tissue, prostate, breast, ovaries, testicles or other reproductive organs, skin, thyroid, blood, lymph nodes, kidney, liver, pancreas, and brain or central nervous system. In one embodiment, the recombinant HCMV virus of the invention is used for the prevention, management, treatment or amelioration of lung cancer, prostate cancer, ovarian cancer, melanoma, bone cancer or breast cancer.

In a preferred embodiment, the polynucleotide sequence encodes an antigenic polypeptide of a protein known to be associated with the development of cancer in animals (preferably humans).

Among the tumor-associated antigens (TAA) that can be used in the recombinant HCMV of the invention are: bullous pemphigoid antigen 2, prostate mucin antigen (PMA) (Beckett and Wright, 1995, Int. J. Cancer 62: 703-710), tumor associated Thomsen-Friedenreich antigen (Dahlenborg et al., 1997, Int. J Cancer 70: 63-71), prostate-specific antigen (PSA) (Dannull and Belldegrun (1997) Br. J. Urol. 1: 97-103), luminal epithelial antigen (LEA 135) of breast carcinoma and bladder transitional cell carcinoma (TCC) (Jones et al., 1997, Anticancer Res. 17: 685-687), cancer-associated serum antigen (CASA) and cancer antigen 125 (CA 125) (Kierkegaard et al., 1995, Gynecol. Oncol. 59: 251-254), the epithelial glycoprotein 40 (EGP40) (Kievit et al., 1997, Int. J. Cancer 71: 237-245), squamous cell carcinoma antigen (SCC) (Lozza et al., 1997 Anticancer Res. 17: 525-529), cathepsin E (Mota et al., 1997, Am. J Pathol. 150: 1223-1229), tyrosinase in melanoma (Fishman et al., 1997 Cancer 79: 1461-1464), cell nuclear antigen (PCNA) of cerebral cavernomas (Notelet et al., 1997 Surg. Neurol. 47: 364-370), DF31MUC1 breast cancer antigen (Apostolopoulos et al., 1996Immunol. Cell. Biol. 74: 457-464; Pandey et al., 1995, Cancer Res. 55: 4000-4003), carcinoembryonic antigen (Paone et al., 1996, J. Cancer Res. Clin. Oncol. 122: 499-503; Schlom et al., 1996 Breast Cancer Res. Treat. 38: 27-39), tumor-associated antigen CA 19-9 (Tolliver and O'Brien, 1997, South Med. J. 90: 89-90; Tsuruta et al., 1997 Urol. Int. 58: 20-24), human melanoma antigens MART-1/Melan-A27-35 and gp 100 (Kawakami and Rosenberg, 1997, Int. Rev. Immunol. 14: 173-192; Zajac et al., 1997, Int. J Cancer 71: 491-496), the T and Tn pancarcinoma (CA) glycopeptide epitopes (Springer, 1995, Crit. Rev. Oncog. 6: 57-85), a 35 kD tumor-associated autoantigen in papillary thyroid carcinoma (Lucas et al., 1996 Anticancer Res. 16: 2493-2496), KH-1 adenocarcinoma antigen (Deshpande and Danishefsky, 1997, Nature 387: 164-166), the A60 mycobacterial antigen (Maes et al., 1996, J. Cancer Res. Clin. Oncol. 122: 296-300), heat shock proteins (HSPs) (Blachere and Srivastava, 1995, Semin. Cancer Biol. 6: 349-355), and MAGE, tyrosinase, melan-A and gp75 and mutant oncogene products (e.g., p53, ras, and HER-2/neu (Bueler and Mulligan, 1996 Mol. Med. 2: 545-555; Lewis and Houghton, 1995 Semin. Cancer Biol. 6: 321-327; Theobald et al., 1995 Proc. Nat'l. Acad. Sci. USA 92: 11993-11997), the EphA2 (Coffman et al., 2003, Cancer Res. 63: 7907-12), and EphA4 (Cheng et al., 2002, Cytokine Growth Factor Rev. 13: 75-85).

In another preferred embodiment, the engineered recombinant HCMV comprises a polynucleotide sequence which is at least 60% identical, or at least 70% identical, or at least 75% identical, or at least 80% identical, or at least 85% identical, or at least 90% identical, or at least 95% identical, or at least 99% identical to a polynucleotide sequence encoding an antigenic polypeptide of a tumor associated antigen (examples are provided supra). In addition, the polynucleotide sequence may encode a full-length antigenic polypeptide or an immunogenic fragment thereof.

7. pp65-Antigen Fusion Protein

A preferred embodiment comprises a fusion protein of pp65 (UL83) and one or more antigenic and/or immunogenic peptide. pp65 is a nonessential CMV matrix protein that is internalized in the cells and delivered into the cytosol at the same time as the virion, very shortly after infection. Without being bound by theory, pp65 can be used whole or in the form of one or more fragments; the peptide fragments which make up the fusion protein preferably have a length of greater than, or equal to, 9 amino acids and cover different HLA class I restrictions. pp65 is the immunodominant protein: 70-90% of all CMV-specific CTL recognized this protein. It has been shown that a peptide of protein pp65 of a length greater than 9 amino acids can be internalized by a presenting cell and presented to a CD8+ specific T-line by a class I MHC molecule. The fusion protein can also enter, via antigen-specific uptake, specific B cells, which in turn are able to present the epitopes both of the heterologous antigenic peptide and of pp65 in the context of MHC class II. In addition, it is also possible for portions of the fusion protein to be presented by professional antigen-presenting cells (APC) in the context of MHC class II. In both cases, the result is efficient stimulation of the T-helper (T_{H}) cell response both to the pp65 and to the antigenic peptide. These T_{H} cells are able to stimulate antigen specific B-cells, which present peptides of pp65 and the antigenic peptide in the context of MHC class II, to form neutralizing antibodies both homologously and heterologously. In addition, the pp65-antigen fusion protein can be introduced by exogenous loading onto the MHC class I pathway. This achieves a stimulation of the CTL response to both pp65 and the antigenic peptide.

The polypeptide sequence of IE1, which is a major regulatory protein of the viral cycle, is known to elicit a CTL response (Kern et al., 1999, J. Virol. 73: 8179-84). Without being bound by any particular theory, production of IE1 during infection with the recombinant HCMV of the invention allows the induction of memory CD4+ helper T-cells, which are capable of cooperating with the induction of cytotoxic CD8+ T-cells and with the production of antibody against both pp65 and the heterologous antigenic peptide. By using an infection competent recombinant HCMV the IE1 protein will be expressed and a full CTL response to be mounted. It is known that peptides of the IE1 protein are in some cases presented by different MHC class I molecules then are peptides of pp65. Thus, the presence of further CTL epitopes from IE1 will ensure that inoculated subjects who express different MHC class I molecules are able to generate CTL against the heterologous peptide as well. As the IE1 polypeptide plays a role in the development and diversity of the CTL response it is specifically contemplated that the pp65-antigen fusion protein of present invention may also comprise part or all of the IE1 polypeptide. In one embodiment the nucleotide sequence encoding the E1 portion of the pp65-fusion protein is provided in addition to the genomic IE1 nucleotide sequence.

In one embodiment, the heterologous nucleotide sequence is added to the complete HCMV genome. In a specific embodiment, HCMV is engineered so that the heterologous nucleotide sequence is expressed a functional fusion with the endogenous pp65 protein. Furthermore, the pp65-antigen fusion protein of the invention is under the control of the endogenous pp65 promoter. In other embodiments the heterologous nucleotide sequence is fused with pp65 in the correct reading frame and further is operably linked to a heterologous promoter. It is specifically contemplated that the pp65-antigen fusion may comprise part or all of the endogenous pp65 protein. It is further contemplated that the pp65-antigen fusion may comprise part or all of an additional copy of pp65 protein, such that the recombinant HCMV of the invention retains a non-substituted genomic pp65 gene in addition to the pp65-antigen fusion.

In still other embodiments the heterologous nucleotide sequence is operably linked directly to a pp65 promoter, which, may be derived from the endogenous pp65 promoter, or be an additional copy of the pp65 promoter.

Construction of a gene that encodes a fusion protein requires routine methods and techniques. Ausubel, F. M., et al., 1998, Current Protocols in Molecular Biology, John Wiley & Sons; Sambrook, et al., 2001, Molecular Cloning: A Laboratory Manual, 3nd Edition. A skilled artisan must link two nucleotide sequences so that the open reading frame is maintained. Using the appropriate restriction sites, one can clone the nucleic acid sequence of one of the polypeptides to be fused (e.g., the heterologous nucleotide sequence) into a vector. Subsequently, the nucleic acid sequence of the second protein (e.g., the viral protein) may also be cloned into the same vector in a manner to maintain the open reading frame and link the nucleotide sequences of both proteins. This is done for example, by appropriately utilizing restriction sites and enzymes. Thereafter, PCR is used to amplify the sequence. The reaction is then digested and a sufficient amount of sequence is transfected into an expression system (e.g., E. coli).

The heterologous polypeptide may be fused or linked to either the amino terminus or the carboxyl terminus of the viral protein. A preferred embodiment of the claimed invention pertains to a HCMV viral protein fused with an immunogenic and/or antigenic peptide. In particular, the preferred embodiment of the invention relates to the pp65-peptide fusion protein. The pp65 viral protein is derived from HCMV. A description of how to construct the fusion protein is discussed throughout the specification and in particular in Examples 1 and 2. Although Example 2 illustrates the specific instructions of how to make the pp65-NS3-NS4 fusion protein, these methods and materials can be adapted to make the fusion protein from any viral protein and desired polypeptide.

8. Construction of a Virus that Expresses the Fusion Protein

Construction of a virus that expresses a fusion protein as described herein, also requires well known molecular cloning techniques. Ausubel, F. M., et al., 1998, Current Protocols in Molecular Biology, John Wiley & Sons; Sambrook, et al., 2001, Molecular Cloning: A Laboratory Manual, 3nd Edition. A skilled artisan can combine the fusion protein's nucleic acid with an HCMV.

As is known in the art and discussed herein, the novel heterologous nucleotide sequence is inserted into the HCMV viral genome using, for example, homologous recombination techniques. The insertion is generally made into a gene, which is not essential for replication in cell culture in nature, for example the pp65 (UL83) gene. Or in a gene that can be compensated for by a complementing cell line (e.g. IE1, see Mocarski et al., 1996, PNAS 93: 11231). The insertion can be made such that the novel heterologous nucleotide sequence generates a fusion protein (e.g., recombined into an endogenous gene in the correct reading frame). It is also specifically contemplated that the novel DNA may be inserted into a non-essential non-coding region so as to leave the endogenous HCMV genes unaffected.

Recombinant HCMV DNA is derived by co-transfecting a plasmid containing the novel heterologous nucleotide sequence, or analogs or fragments thereof, and a selectable marker such as gpt or another marker such as β-galactosidase or GFP along with HCMV DNA, or bacmid in primary fibroblast cells, or other cell lines known to be permissive for growth of HCMV. In addition to the viral DNA, plasmids could be transfected in cells (either prior to, during or following transfection of the plasmid) that are infected with HCMV. Recombinant viruses are selected by growth in media containing mycophenolic acid or identified by blue plaque phenotypes after applying a chromogenic substrate such as X-gal. Recombinant viruses are plaque purified and characterized by restriction enzyme analysis and Southern blotting procedures. Plasmid shuttle vectors that greatly facilitate the construction of recombinant viruses have been described and such methods are well known in the art, see for example: Spaete and Mocarski, 1987, Proc. Nat. Acad. Sci. 84: 7213-17.

The heterologous nucleotide sequence, or analogs or fragments thereof, may be fused in frame with an endogenous HCMV gene thus, under the control of the endogenous promoter. Alternatively, the novel heterologous nucleotide sequence can be placed under transcriptional control of a promoter such as the HCMV major immediate early promoter, the SV40 early promoter or some other viral or cellular promoter that generates adequate levels of expression. One can make a HCMV which expressions a heterologous protein using known methods in the art and methods detailed in: European Patent application 0 277 773 A1, U.S. Pat. Nos. 5,830,745, 6,713,070, 6,692,954, 5,721,354 and U.S. patent application Ser. No. 10/061,943 the teachings of which are incorporated herein by reference in their entirety. Expression of the polypeptide encoded by the heterologous nucleotide sequence then occurs in cells or individuals that are immunized with the live recombinant virus.

The heterologous nucleotide sequence, or analogs or fragments thereof, may further incorporated a protein cleavage site to allow for the cleavage of the expressed fusion protein and release of the heterologous polypeptide. In one embodiment, the cleavage site is selected such that cleavage of the fusion protein can occur in vivo. A number of protein cleavage site are well known in the art and include, for example, the FMDV 2A cleavage site derived from the Foot-and-Mouth Disease Virus 2A sequence.

The recombinant HCMV of the invention is grown in tissue culture cells. For experiments with mammals, not including humans, cells such as human foreskin fibroblasts or MRC-5 cells are used to propagate the virus. The virus is harvested from cultures of these cells and the isolated recombinant virus is then further studied for its ability to elicit an immune response and provide protection against HCMV infection.

For use in humans, the recombinant virus is produced from an FDA approved cell line in large scale amounts. Such cells include MRC-5 or WI-38 cells (both are primary human diploid fibroblasts). The recombinant virus is generated in the production cell line by transfection of viral DNA or infection with intact virions prepared from recombinant virus isolated from another cell line. The method of transfection should prevent the contamination of FDA approved cells with adventitious agents or contaminants from a non-qualified cell line. HCMV virus produced from the above cell lines will be used to infect progressively larger flasks of tissue culture cells. Infected cells or virions isolated from infected cells will be used as subsequent inoculums. Viable infected tissue culture cells are removed from the tissue culture vessels and added to a 1 to 100 fold (or more) excess of uninfected cells to accomplish progressively larger inoculations. Once an optimal yield is obtained the virus will be harvested from the tissue culture cells. This process can be repeated until a large scale production is achieved. Infected cells will be removed from the tissue culture vessel and disrupted using for example, sonication, dounce homogenization or some combination of the above. The viruses are then isolated from cellular material using centrifugation techniques or other purification techniques (e.g., columns or filters) known in the art. Once the virus is isolated a stabilizing agent is added, such as a carbohydrate or carbohydrate derivative and the virus is then aliquoted and lyophilized.

9. Attenuation of HCMV

The recombinant viruses of the invention can be further genetically engineered to exhibit an attenuated phenotype. In particular, the recombinant viruses of the invention exhibit an attenuated phenotype in a subject to which the virus is administered. Attenuation can be achieved by any method known to a skilled artisan (see for example U.S. Pat. No. 3,959,466, and European Patent Application 0 277 773 A1 the teachings of which are incorporated herein by reference in their entirety). Without being bound by any particular theory, the attenuated phenotype of the recombinant virus can be created, for example, by using a virus that naturally does not replicate well in an intended host, by reduced replication of the viral genome, by reduced ability of the virus to infect a host cell, or by reduced ability of the viral proteins to assemble to an infectious viral particle relative to the wild type strain of the virus, or by modification of the immune response to the virus. The attenuated phenotypes of a recombinant virus of the invention can be tested by any method known to the artisan. A candidate virus can, for example, be tested for its ability to infect a host or for the rate of replication in a cell culture system.

In certain embodiments, the attenuated virus of the invention is capable of infecting a host, is capable of replicating in a host such that infectious viral particles are produced. In comparison to the wild type strain, however, the attenuated strain grows to lower titers or grows more slowly. Any technique known to the skilled artisan can be used to determine the growth curve of the attenuated virus and compare it to the growth curve of the wild type virus.

In certain embodiments, the ability of the attenuated mammalian virus to infect a host is reduced compared to the ability of the wild type virus to infect the same host. Any technique known to the skilled artisan can be used to determine whether a virus is capable of infecting a host.

10. Vaccine Formulation and Administration

The invention encompasses vaccine formulations comprising the engineered recombinant HCMV of the present invention. The recombinant HCMV of the present invention may be used as a vehicle to express foreign epitopes that induce a protective response to any of a variety of pathogens and diseases. In a specific embodiment, the invention encompasses the use of recombinant HCMV or attenuated HCMV viruses that have been modified in vaccine formulations to confer protection against the pathogen or disease represented by the foreign antigen.

The vaccine preparations of the invention encompass multivalent vaccines, including bivalent and trivalent vaccine preparations. The bivalent and trivalent vaccines of the invention may be administered in the form of one HCMV expressing each heterologous antigenic sequence or two or more HCMV each encoding different heterologous antigenic sequences. For example, a first chimeric HCMV expressing one or more heterologous antigenic sequences can be administered in combination with a second chimeric HCMV expressing one or more heterologous antigenic sequences, wherein the heterologous antigenic sequences in the second chimeric HCMV are different from the heterologous antigenic sequences in the first chimeric HCMV.

When used for vaccination, vaccination with the compositions of the invention may be prophylactic vaccination (wherein the vaccine is administered prior to exposure, or anticipated exposure, to the target antigen, e.g., to a subject susceptible to or otherwise at risk of exposure to a disease) and/or immunotherapeutic vaccination (wherein the vaccine is administered after exposure to the target antigen to accelerate or enhance the immune response).

The vaccine preparations of the invention are typically administered intradermally or subcutaneously, but can also be administered in a variety of ways, including orally, by injection (e.g., intradermal, subcutaneous, intramuscular, intraperitoneal and the like), by inhalation, by topical administration, by suppository, using a transdermal patch.

When administration is by injection, the compositions may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

In some instances, multiple preparations of recombinant viruses (e.g., each encoding and expressing a different antigen or immunogenic polypeptide) can be administered.

Typically, an amount of the viral composition will be administered to the subject that is sufficient to immunize an animal against an antigen (i.e., an “immunologically effective dose” or a “therapeutically effective dose”). The effective dose can be formulated in animal models to achieve an induction of an immune response using techniques that are well known in the art. Exemplary doses are 10 to 10⁷ pfu per dose, e.g., 10 to 10⁶ or 10³ to 10⁶ pfu. One having ordinary skill in the art can readily optimize administration to humans (e.g., based on animal data and clinical studies).

In various embodiments, the compositions include carriers and excipients (including but not limited to buffers, carbohydrates, mannitol, proteins, polypeptides or amino acids such as glycine, antioxidants, bacteriostats, chelating agents, suspending agents, thickening agents and/or preservatives), other pharmaceutically acceptable auxiliary substances as required to approximate physiological conditions, such as buffering agents, tonicity adjusting agents, wetting agents and the like, and/or a conventional adjuvant (e.g., Freund's Incomplete Adjuvant, Freund's Complete Adjuvant, Merck Adjuvant 65, AS-2, alum, aluminum phosphate, mineral gels such as aluminum hydroxide, and surface active substances such as lysolecithin, pluronic polyols, polyanions, peptides, oil emulsions, keyhole limpet hemocyanin, and dinitrophenol). It will be recognized that, while any suitable carrier known to those of ordinary skill in the art may be employed to administer the compositions, the type of carrier will vary depending on the mode of administration. Compounds may also be encapsulated within liposomes using well known technology.

The compositions are usually produced under sterile conditions and may be substantially isotonic for administration to hosts. Typically, the compositions are formulated as sterile, and in full compliance with all Good Manufacturing Practice (GMP) regulations of the U.S. Food and Drug Administration.

It is understood that the embodiments described herein are for illustrative purposes only and that various modifications or changes in light thereof will be suggested to persons skilled in the art and are to be included within the spirit and purview of this application and the scope of the appended claims. All publications, patents, and patent applications cited herein are hereby incorporated by reference for all purposes.

### EXAMPLES

The invention is now described with reference to the following examples. These examples are provided for the purpose of illustration only and the invention should in no way be construed as being limited to these examples but rather should be construed to encompass any and all variations which become evident as a result of the teachings provided herein.

### Example 1

### Generation of Recombinant HCMV

This example describes a general protocol for the construction of the recombinant human cytomegalovirus described in the application. The recombinants may include 1) modifications or mutations that result in a virus with a phenotype more suitable for use in vaccine formulations, 2) insertion of one or more heterologous genes or gene fragments encoding a foreign antigen into the endogenous pp65 protein to generate a pp65 fusion protein, 3) insertion of a gene encoding a pp65-antigen fusion operable linked to a promoter in a discrete location from the endogenous pp65 gene, 4) insertion of one or more heterologous genes or gene fragments encoding a foreign antigen operably linked to either the endogenous pp65 promoter or a promoter in a discrete location, and 5) combinations of the above.

Materials and Methods

Handling CMV, and constructing CMV genomic mutants: General methods are known to practitioners of the virology art. See, e.g., Mocarski et al., 1996, Intervirology 39: 320-30; Spaete et al, 1987, Proc. Natl. Acad. Sci. USA 84: 7213-17; Ehsani et al., 2000, J Virol, 74: 8972-79.

Viral DNA Preparation: Viral DNA can be prepared from infected cells using the method of Ehsani et al., 2000, J Virol 74: 8972-9, or other suitable methods.

Propagation of Virus and Cells: Virus and cells are propagated by standard methods. Human CMV is propagated in human fibroblasts, e.g., human foreskin fibroblast (HFs), MRC-5 (ATCC #CCL 171) and others, grown in Dulbecco's minimal essential medium (DMEM) with 10% fetal bovine serum supplemented with amino acids and antibiotics according to standard techniques (Spaete and Mocarski, 1985, J. Virology 56: 135-43). Viral stocks are titered by making several 10-fold dilutions and adding them to HFs grown to confluency in T-25 flasks. Flasks are incubated ˜7 days until plaques are visible.

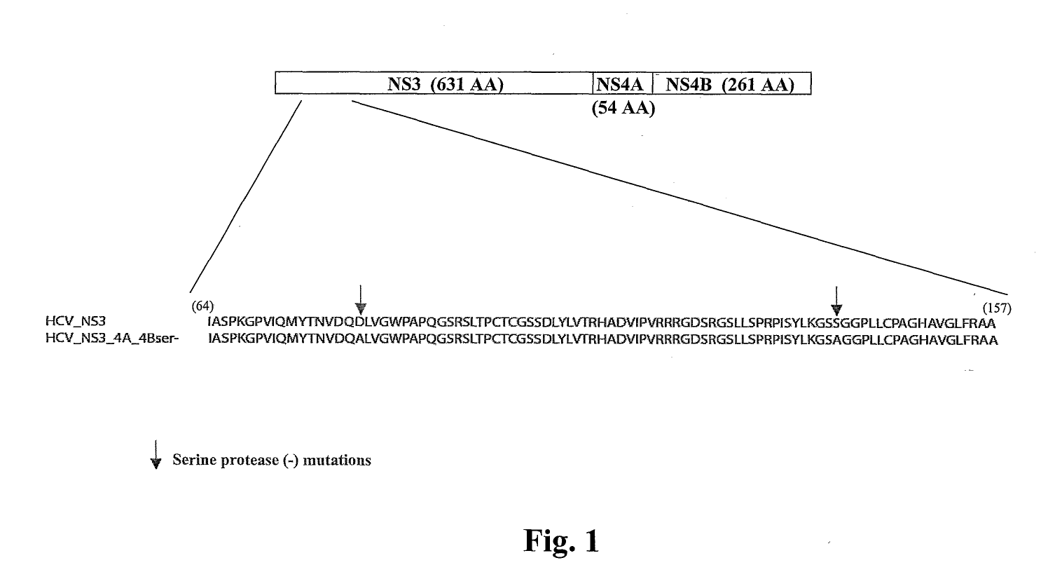

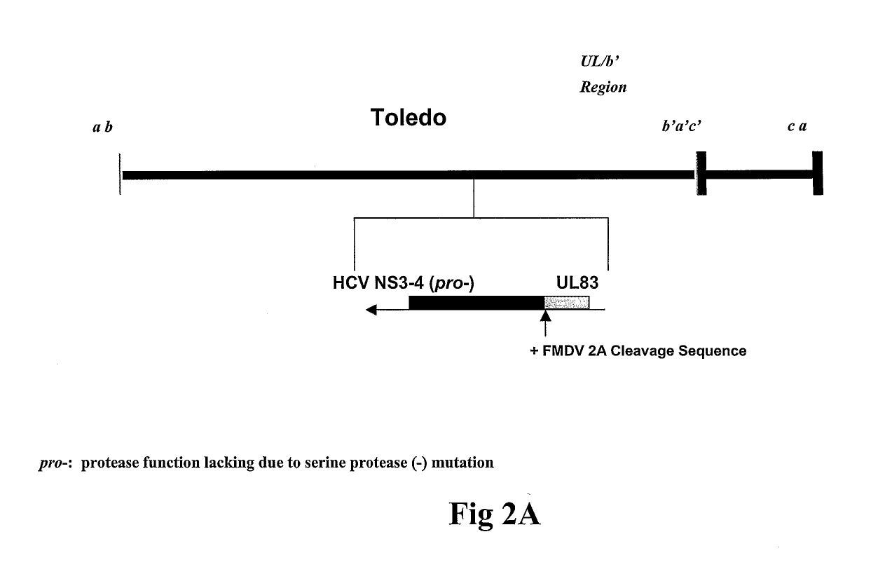

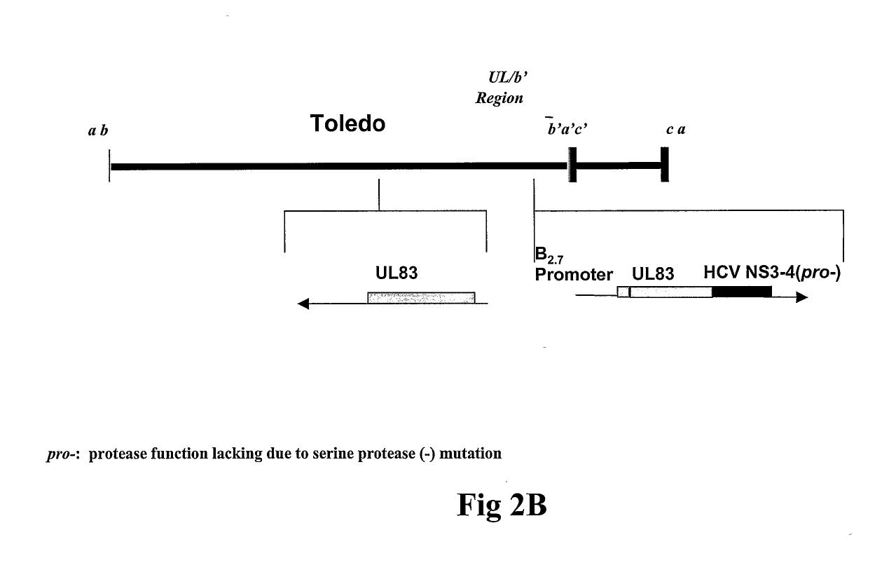

Insertion of polynucleotide Encoding a Heterologous Antigen into the CMV Genome: This example describes a general protocol for the construction of the recombinant cytomegaloviruses described in the application and more specifically for the recombinant HCMV described in FIG. 2, containing the HCV antigen detailed in FIG. 1.

In one embodiment the HCMV UL83 gene is to be fused with a polynucleotide sequence encoding one or more heterologous antigen genes by constructing a targeting plasmid vector. The targeting vector contains the polynucleotide encoding the heterologous antigen operably linked UL83 sequences and flanked by sequence from the viral gene (typically at least 300, and often at least 500, base pairs of viral sequence is present at each end of the insert, i.e., the 5′ and 3′ ends). In constructing the targeting vector, one suitable approach is to clone the pp65 viral gene including flanking sequences into a (plasmid) vector and insert an expression cassette encoding the entire heterologous antigen gene or a fragment thereof. It will be apparent to one of ordinary skill that these constructs can be prepared using routine molecular biological techniques, e.g., insertion of synthetic, amplified or subcloned gene fragments of interest into existing restriction sites in a target sequence; introduction of additional restriction sites using synthetic linkers, site-directed mutagenesis; and the like.

CMV recombinants are generated by co-transfection of the targeting vector and wild type CMV viral DNA or chimeric CMV viral DNA into permissive cells of the appropriate species (e.g., human dermal fibroblasts, rhesus dermal fibroblasts, murine 3T3 fibroblasts) using calcium phosphate transfection or other methods well known in the art. Cells containing recombinant virus are purified from wild type infected cells by selection with antibiotic, colormetric selection, or the like. Selected infected cells are then subjected to plaque purification by standard techniques to obtain a pure recombinant virus preparation.

The purified virus preparation is then combined with a suitable carrier (e.g., physiological saline containing a stabilizer) for administration to a host animal or cell line.

Recombinant virus can also be made by cotransfecting the targeting vector with cosmids of HCMV or cloned bacterial plasmids containing the entire recombinant HCMV genome (called a bacmid). Designed appropriately, only the correct thing comes out; there's no need to screen or rely on recombination to make the correct construct. For cosmid transfection, see Kemble et al., 1996, J Virol. 70: 2044-8, for bacmids see Hahn G, et al., 1999, J Virol. 73: 8320-9)

Determination of pp65-fusion protein Expression Levels: Expression levels are determined by infecting cells in culture with a recombinant virus of the invention and subsequently measuring the level of protein expression by, e.g., Western blot analysis or ELISA using antibodies specific to either the pp65 or preferably the heterologous gene product, or measuring the level of RNA expression by, e.g., Northern blot analysis using probes specific to the heterologous sequence. Similarly, expression levels of the heterologous sequence are determined by infecting an animal model and measuring the level of protein expressed from the heterologous sequence of the recombinant virus of the invention in the animal model. The protein level is measured by obtaining a tissue sample from the infected animal and then subjecting the tissue sample to Western blot analysis or ELISA, using antibodies specific to the gene product of the heterologous sequence. Further, if an animal model is used, the titer of antibodies produced by the animal against the gene product of the heterologous sequence is determined by any technique known to the skilled artisan including but not limited to, ELISA.

### Example 2

### Generation and Characterization of Recombinant HCMV Expressing a HCV NS3-4A-4B (pro-)-UL83 fusion

A recombinant HCVM virus was constructed expressing the HCV NS3-4A-4B gene product. Specifically a protease deficient variant (see FIG. 1). In this case the recombinant virus expressed the HCV gene from the endogenous native UL83 gene (see FIG. 2A) however, also contemplated is the expression of a HCV NS3-4A-4B (pro-)-UL83 fusion from a location eptoptic to the native locus (see FIG. 2B). The genomic organization of the recombinant virus was confirmed by Southern blot analysis, and the expression of the HCV NS3 protein was shown by Western blot analysis.

Materials and Methods

Construction of HCMV Expressing a HCV NS3-4A-4B (ser-)-pp65 fusion: The technique used for rescue of recombinant HCMV by overlapping cosmids was performed essentially as previously described (Kemble et al., 1996). To facilitate the construction of Toledo 2A HCV by overlapping cosmids, a cosmid containing the 2A HCV sequences (NS3-4A-4B protease deficent (pro-), see FIG. 1) inserted at the 3′ end of the UL83 gene (encodes the pp65 protein) was constructed (see FIG. 2) using homologous recombination in yeast as follows: The HCV NS3-4A-4B (pro-) DNA was synthesized (DNA 2.0, Inc.) with the Foot-and-Mouth Disease Virus 2A cleavage sequence (FMDV 2A) 5′ of the NS3 sequences and UL83 sequences from the regions adjacent to the desired insertion point flanking the ends of the HCV sequences and cloned into the vector pDrive resulting in pDriveG00343. A kanamycin gene with flanking Pme I sites was then cloned at the 3′ end of the HCV sequences in pDriveG00343 resulting in pDriveG00343kan. The presence of the FMDV 2A cleavage site allows processing of the fusion protein into separate NS3 and pp65 proteins.

S. cerevisiae CGY2570 cells were transformed with pDriveG00343kan and Tol 158y, a construct with Toledo cosmid sequences from the UL83 region carried on a vector composed of pACYC184 (Rose, 1988) and yeast ARS CEN HIS sequences (Christianson et al., 1992) and the resulting transformed yeast cells were selected for kanamycin expression using G418. Cosmid DNA was prepared from G418 resistant colonies and electroporated into Top 10 electrocompetent bacteria (Invitrogen). Cosmid DNA, prepared from chloramphenicol resistant bacteria, was analyzed by restriction enzyme digestion and nucleotide sequence determination to confirm that the 2A HCV sequences had recombined correctly into the UL83 locus.

The DNA from one transformant with the correct structure, Tol 158y 2A HCV kan, was digested with Pme I, which releases the kanamycin insert, and recircularized by treatment with T4 DNA ligase to generate Tol 158y 2A HCV. Combination of Tol 158y 2A HCV with 7 Toledo cosmids from the rest of the HCMV genome, each digested with PacI to release their Toledo inserts, resulted in an overlapping set of clones representing the Toledo genome with 2A HCV insertion at the 3′ end of the UL83 gene. This mixture was used to transfect human lung fibroblast cells by the calcium phosphate method resulting in the Toledo recombinant virus Toledo 2A HCV.

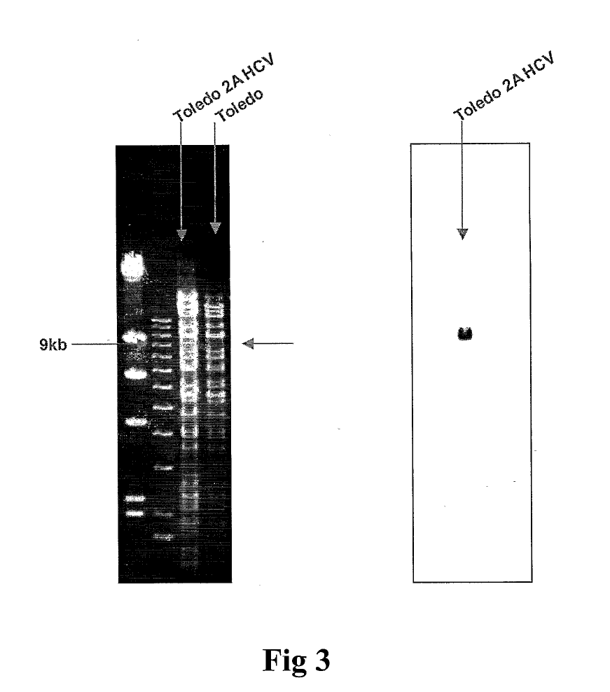

Southern Blot Analysis of Toledo 2A HCV: Viral DNA was harvested from human lung fibroblast infected with either Toledo 2A HCV or Toledo virus. After digestion with Bam HI, the viral DNAs were subjected to Southern blot analysis using a fluorescently labeled probe specific for the inserted HCV sequences. As predicted from the Toledo and HCV inserted sequences, a 9 kb fragment which hybridized to the HCV probe appeared exclusively in the Toledo 2A HCV viral DNA (See FIG. 3).

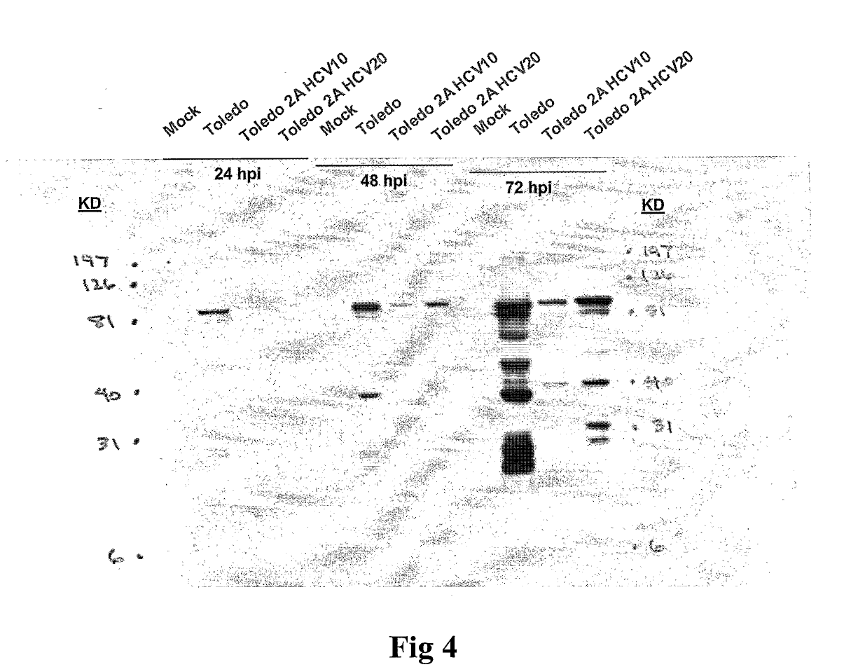

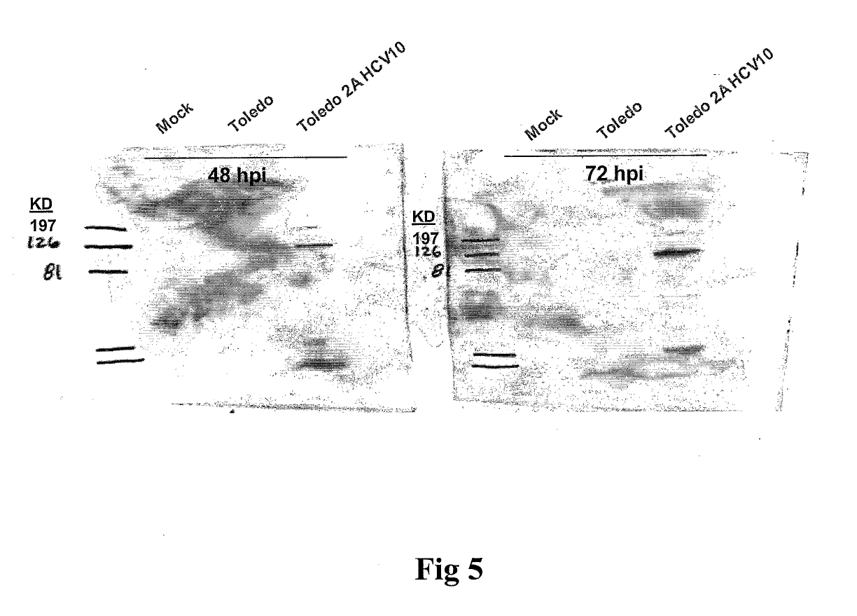

Western Blot Analysis of Toledo 2A HCV: Human lung fibroblast were infected with either Toledo, Toledo 2A HCV or mock infected. At 24, 48 and 72 hours post infection (hpi), protein lysates were prepared by washing cell monolayers with ice cold PBS followed by lysis in SDS PAGE loading buffer containing freshly added DTT (final concentration 25 micromolar). Lysates were then immediately boiled for 5 min and stored at −20 degrees. The lysates were then separated by SDS-PAGE and the protein transferred to a membrane. The membrane was then probed with anti-pp65 (FIG. 4), or anti-NS3 (FIG. 5).

FIG. 4 demonstrates the presence of pp65 specific bands in cells infected with either the wild type Toledo strain or the recombinant Toledo 2A HCV strains but not the mock infected cells. The pp65 protein made in the recombinant is cleaved at the FMDV cleavage site, and retains the anti IRF3 activity of wild type pp65 (data not shown). At 72 hours post infection, a anti-NS3-specific protein band is present exclusively in the Toledo 2A HCV lysates (FIG. 5).

### Example 3

### Primate Model Studies

It is a goal of this invention to provide a recombinant HCMV expressing a pp65 polypeptide or fragment thereof fused to a heterologous polypeptide. In particular, the present invention encompasses immunogenic preparations (e.g., vaccine). This example details the primate system used to test the efficacy of the recombinant HCMV of the invention to modulate disease in an animal. This proof of concept can be expanded to encompass recombinant HCMV vectors to provide an immune response to numerous other pathogens, viruses and cancer antigens as described supra.

Hepatitis C virus (HCV) is one of the major blood-borne viruses that infects more then 100 million people world-wide. The CD8⁺ T-cell response is thought to be important for the control of HCV. Thus, the recombinant HCMV of the invention expressing a pp65-HCV antigen is an ideal immunogenic preparation to modulate and/or prevent the progression of disease mediated by HCV.

HCMV is a well known, ubiquitous infection in many species including primates. Humans and chimpanzees are naturally infected by HCMV and chimpanzee cytomegalovirus (CCMV), respectively. These two viruses are distinct, yet immunologically cross-reactive and appear to have a similar natural history. HCMV can replicate in primary chimpanzee fibroblasts (Perot et al., 1992, J. Gen. Virol. 73: 3281-84) demonstrating the close relatedness of the human and primate viruses. In addition, naturally infected humans and chimpanzees have easily detectable CMV specific CD8⁺ T cells circulating in peripheral blood; a large proportion of these CMV specific CD8⁺ cells react to the pp65 (UL83) protein demonstrating the similarities in the immune response between humans and chimpanzees. It is also know that both HCMV and CCMV, like all herpes viruses, establish a latent or persistent infection in the host and the immune response remains high throughout much of the life of a healthy adult.

Materials and Methods

Generation of Recombinant HCMV Expressing A pp65-HCV N3-N4 Fusion Protein: Recombinant HCMV are produced that express part or all of both the HCV NS3 (see FIG. 1) and NS4 proteins fused to pp65, the backbone of which will be derived from the Toledo strain (see FIG. 2). These HCV genes are fused to the human UL83 gene segment encoding pp65 and placed under the control of an HCMV gene including either UL83 endogenous promoter or another strong regulatory element such as another HCMV promoter like the β2.7 gene promoter or a non-HCMV promoter such as the SV40 viral promoter. These constructs are placed in the endogenous position of UL83 or in an ecotopic site by methods well known in the art and described supra. These recombinants are evaluated in vitro for their ability to express the HCV products prior to being used to immunize animals. A stock of the selected recombinant is produced and tested for sterility prior to use in animals.

Determination of pp65-HCV N3-N4 Fusion Protein Expression Levels: Expression levels are determined in an appropriate cell line as described supra.

Inoculation Procedures and Schedule: CCMV seropositive/HCV negative chimpanzees will be inoculated subcutaneously with the recombinant virus. Immunized animals will be bled at routine intervals (approximately weekly) for several weeks following immunization. The blood will be analyzed by PCR and standard virology assays for evidence of recombinant HCMV replication. The blood and sera will be analyzed for evidence of antibodies to HCMV as well as the HCV antigens. Cells derived from the blood will be assayed by intracellular cytokine assays as well as routine cell killing assays for evidence of a cellular immune response to the HCV antigens. (For example see Rollier et al., 2004, J Virol. 78: 187-96, Erickson et al., 1993, J. Immunol. 151: 4189-99).

Stocks of the recombinant virus are grown and titered on human fibroblast cells. Approximately 10⁷ PFU of the recombinant virus are delivered one time to each of 2-4 CMV seropositive, HCV naïve chimpanzees intramuscularly. Blood is drawn on the day of vaccination as well as at various intervals post infection. Precursor frequencies of CD4 and CD8 cells specific for HCMV and HCV are measured as are antibody responses to the HCV antigen. An increase in CMV specific T cells is indicative of active viral replication and eliciting HCV specific T cells indicates that the recombinant virus effectively delivered the appropriate antigen in a manner recognized by the immune system. To determine the precursor frequencies of these cells, both intracellular cytokine staining as well as ELISPOT assays are performed. Additionally, needle biopsies of organs such as liver can be obtained allowing the measurement of HCV specific T cells in this target organ.

The quantity of HCMV in various tissues, including blood and biopsied material from the injection site, and fluids such as saliva and urine are assessed using sensitive PCR based assays that can measure the amount of viral DNA or assays that can evaluate the quantity of infectious virus in a specimen. Samples such as blood, saliva and urine are obtained at least once every two weeks for 28 weeks post vaccination whereas skin and liver biopsies are performed less frequently.

Following the initial studies to determine whether the vaccine can elicit an HCV specific T cell response in HCMV seropositive and HCV naïve animals, studies are performed to determine whether this vector can elicit a functional immune response that can control or eliminate HCV infection in a chronically infected animal. To perform these experiments, CMV serpositive, persistently HCV infected chimpanzees are immunized with the CMV vector containing the HCV antigen or the CMV vector alone without antigen. The animals are then assessed for many of the parameters from the earlier study and in addition, for HCV viral load and HCV genome structure. These latter two parameters are important to show that the immune response elicited by the CMV vector (HCV antigen) can reduce the HCV viral load in these animals and not drive the evolution of HCV genomes that would be resistant to the immune response. These studies represent a therapeutic type of vaccine strategy.

In addition, studies in which the CMV (HCV vector) is used to immunize CMV serpositive, HCV naïve animals as outlined in the first series of experiments can also be performed. Following establishment of an immune response, these animals could be directly challenged with HCV and monitored for HCV viral load and other markers of HCV infection. These studies are used to evaluate the prophylactic role of the CMV vector in this model system.

Methods and protocols for performing vaccine studies using the chimpanzee animal model are well known in the art. See for example Shoukry et al., 2004, J Immunol. 172, 483-92, and Woollard et al., 2003, Hepatology 38, 1297-306.

Determination of CD4+ and CD8+ Responses: CD4+ T cell responses are monitored upon in vitro recall of peripheral or splenic mononuclear cells with the antigen used to immunized animals. Lymphoproliferative responses as well as cytokine inductions (Th1/Th2 balance) are measured (for a review see Jenkins, 2001, Annu Rev Immunol. 19: 23-45). CD8+ T cell responses are evaluated (ex vivo or upon re-stimulation of mononuclear cells) either using 1) a standard Chromium release assay which directly measures antigen specific lytic activity (Brossart et al., 1997, Blood, 90: 1594-99) or using IFNγELISPOT or ICC (intracellular cytokine) assays that both measure the ability of CD8 cells to be stimulated by a 9 mer peptide specific for the antigen versus an irrelevant 9 mer peptide (Carvalho et al., 2001, J. Immunol. Methods, 252: 207-18) for IFNγELISPOT and (King et al., 2001, Nature Medicine, 7: 206-14) for ICC. Other assays to identify functional antigen-specific CD8+ T cell responses are well known in the art, see for example, He et al., 1999, Proc. Natl. Acad. Sci. USA 96: 5692-7, He et al., 2001, Immunol. 14: 59-69, and Lee et al., 1999, Nat. Med. 5: 677-85.

Intracellular Cytokine Assay: Innate immune responses are monitored by measuring the levels of pro-inflammatory (IL-6, TNFα) and/or anti-viral (type I interferons) cytokines in the serum of immunized animals or upon in vitro antigen specific re-stimulations. The early stimulation of innate immunity is also evaluated by assessing the ex vivo activation status of antigen presenting cells (monocytes, dendritic cells) and NK cells that are derived from recently immunized animals (Krishnan et al., 2001, J. Immunol. 166: 1885-93).

⁵¹Cr-Release Assay: ⁵¹Cr-release assays are performed as described (Liu et al., 1999, J. Virol. 73: 9849-57). CTL activity is calculated as the percentage of specific ⁵¹Cr release using the following equation: % specific killing=(sample release-spontaneous release)/(maximal release-spontaneous release)×100%.

HCV and HCMV Antibody Assays: An appropriate method is used to collect the desired sample (e.g., serum, nasal secretion, tissue). After collection and treatment of the sample, detection of total antibodies is realized with ELISAs e.g.: Capture ELISA for detection of any specific immunoglobulin (Ig) subtype (e.g., IgA, IgE, IgG, IgM) Total Igs are captured with anti-chimp Ig polyclonal affinity purified Ig immobilized on microtiter plates and subsequently detected using a different polyclonal anti-chimp Ig affinity purified Ig coupled to peroxidase or other detection molecule. Purified chimp Ig is used as a standard to allow the quantification of Ig in the collected samples.

Alternatively, specific anti-HCV and/or HCMV-pp65 antibodies are determined by altering the conditions of the capture ELISA as follows: Specific anti-HCV and/or HCMV-pp65 antibodies are captured with HCV and/or HCMV-pp65 polypeptide antigens immobilized/coated on microtiter plates and detected using a polyclonal anti-chimp Ig affinity purified Ig coupled to peroxidase or other detection molecule.

HCMV Replication Assay: In another suitable assay, blood is assayed for viral DNA by PCR using CMV specific primers. For example, in one embodiment, DNA is purified from plasma (e.g., using commercially available kits from Qiagen, Calif.), then used as template for nested PCR with primers able to amplify the HCMV N3 and/or N4 genes and/or the pp65 component of the fusion protein.

### Example 4

### Clinical Trials

The vaccines described herein can be used to induce a therapeutic or protective immune response in a patient and in methods for treating diseases such as those described supra. Such methods include administering to a patient a therapeutically effective amount of a vaccine of the invention. A therapeutically effective amount of the vaccine is an amount sufficient to elicit a therapeutic or protective immune response, such as inducing the formation of antibodies and/or other cellular immune responses (e.g., the induction of helper T cells, cytotoxic killer T-cells, anomalous killer cells (AK cells) and/or antibody-dependent cytotoxic cells).

Doses, methods of administrating, and suitable pharmaceutical carriers can be determined readily by the skilled artisan. For example, appropriate doses can be extrapolated from dose-response studies in animals, including non-human primates. An appropriate immunization schedule can be determined by a skilled artisan but generally depends upon the susceptibility of the host or patient to immunization with the vaccine and is typically continued until sufficient antibody is detectable in whole serum. The vaccines can be administered in a variety of different ways including, for example, by oral, intranasal, intraperitoneal, intravenous, intramuscular, subcutaneous, subdermal and transdermal methods. It has been found in some instances that although similar immunologic responses are generated by either intraperitoneal or subcutaneous administration that the latter form of administration is capable of inducing higher T-cell responses.

Methods utilizing the recombinant HCMV of the invention as a vaccine and to assess acquired immunity to the heterologous antigen can be determined using known methods in the art and methods detailed in: Just et al., 1975, Infection 3: 111-4; Starr et al., 1981, J. Infect. Dis. 143: 585-9; Quinnan et al., 1984, Ann. Intern. Med. 101: 478-83; Plotkin et al., 1984, Lancet 1: 528-30; Plotkin et al., 1991, Ann. Intern. Med. 114: 525-31; Adler et al., 1995, J. Infect. Dis. 171: 26-32 [published erratum appears in 1995, J. Infect. Dis. 171: 1080]; Adler et al, 1998, Pediatr. Infect. Dis. J. 17: 200-6; the teachings of which are incorporated herein by reference in their entirety.

Vaccines of the invention that have been tested in in vitro assays and animal models may be further evaluated for safety, tolerance, immunogenicity, infectivity and pharmacokinetics in groups of normal healthy human volunteers, including all age groups. In a preferred embodiment, the healthy human volunteers are infants at about 6 weeks of age or older, children and adults. The volunteers are administered intranasally, intramuscularly, intravenously, subcutaneously or by a pulmonary delivery system in a single dose of a recombinant virus of the invention and/or a vaccine of the invention. Multiple doses of virus and/or vaccine of the invention may be required in seronegative volunteers. Multiple doses of virus and/or vaccine of the invention may also be required to stimulate local and systemic immunity. A recombinant virus of the invention and/or a vaccine of the invention can be administered alone or concurrently with other vaccines or therapeutic agents.

In a preferred embodiment, double-blind randomized, placebo-controlled clinical trials are used. In a specific embodiment, a computer generated randomization schedule is used. For example, each subject in the study will be enrolled as a single unit and assigned a unique case number. Multiple subjects within a single family will be treated as individuals for the purpose of enrollment. Parent/guardian, subjects, and investigators will remain blinded to which treatment group subjects have been assigned for the duration of the study. Serologic and virologic studies will be performed by laboratory personnel blinded to treatment group assignment. The serologic and virologic staff are separate and the serology group will be prevented from acquiring any knowledge of the culture results.

Each volunteer is preferably monitored for at least 12 hours prior to receiving the recombinant virus of the invention and/or a vaccine of the invention, and each volunteer will be monitored for at least fifteen minutes after receiving the dose at a clinical site. Then volunteers are monitored as outpatients on days 1-14, 21, 28, 35, 42, 49, and 56 postdose. In a preferred embodiment, the volunteers are monitored for the first month after each vaccination as outpatients. All vaccine related serious adverse events will be reported for the entire duration of the trial. A serious adverse event is defined as an event that 1) results in death, 2) is immediately life threatening, 3) results in permanent or substantial disability, 4) results in or prolongs an existing in-patient hospitalization, 5) results in a congenital anomaly, 6) is a cancer, or 7) is the result of an overdose of the study vaccine. Serious adverse events that are not vaccine related will be reported beginning on the day of the first vaccination (Day 0) and continue for 30 days following the last vaccination. Non-vaccine related serious adverse events will not be reported for 5 to 8 months after the 30-day reporting period following the last vaccination. A dose of vaccine/placebo will not be given if a volunteer has a vaccine-related serous adverse event following the previous dose. Any adverse event that is not considered vaccine related, but which is of concern, will be discussed by the clinical study monitor and the medical monitor before the decision to give another dose is made.

Blood samples are collected via an indwelling catheter or direct venipuncture (e.g., by using 10 ml red-top Vacutainer tubes) at the following intervals: (1) prior to administering the dose of the recombinant virus of the invention and/or a vaccine of the invention; (2) during the administration of the dose of the recombinant virus of the invention and/or a vaccine of the invention; (3) 5 minutes, 10 minutes, 15 minutes, 20 minutes, 30 minutes, 1 hour, 2 hours, 4 hours, 8 hours, 12 hours, 24 hours, and 48 hours after administering the dose of the recombinant virus of the invention and/or a vaccine of the invention; and (4) 3 days, 7 days 14 days, 21 days, 28 days, 35 days, 42 days, 49 days, and 56 days after administering the dose of the recombinant virus of the invention and/or a vaccine of the invention. In a specific embodiment, a total of 5 blood draws (3-5 ml each) are obtained, each just prior to the first, third and booster doses and approximately one month following the third dose and booster dose of administration of the vaccine or placebo. Samples are allowed to clot at room temperature and the serum is collected after centrifugation.

Sera are tested for strain-specific antibody levels against the virus of the invention. Other indicators of immunogenicity such as IgG, IgA, or neutralizing antibodies are also tested. Serum antibody responses to one or more of the other vaccines given concurrently may be measured. The amount of antibodies generated against the recombinant virus of the invention and/or a vaccine of the invention in the samples from the patients can be quantitated by ELISA. T-cell immunity (cytotoxic and helper responses) in PBMC can also be monitored.

The concentration of antibody levels in the serum of volunteers are corrected by subtracting the predose serum level (background level) from the serum levels at each collection interval after administration of the dose of recombinant virus of the invention and/or a vaccine of the invention. For each volunteer the pharmacokinetic parameters are computed according to the model-independent approach (Gibaldi et al., eds., 1982, Pharmacokinetics, 2nd edition, Marcel Dekker, New York) from the corrected serum antibody or antibody fragment concentrations.

It is understood that the examples and embodiments described herein are for illustrative purposes only and that various modifications or changes in light thereof will be suggested to persons skilled in the art and are to be included within the spirit and purview of this application and scope of the claims. Each and every publication, patent, and patent application cited herein are hereby incorporated by reference in their entirety for all purposes. In particular, U.S. Provisional Application No. 60/583,188 filed Jun. 25, 2004 is incorporated by reference in its entirety for all purposes.

1. A recombinant CMV virus expressing a polynucleotide encoding HCMV pp65 polypeptide or fragment thereof fused to a heterologous-polynucleotide, wherein said heterologous polynucleotide encodes at least one polypeptide.

2. The recombinant CMV virus of claim 1, wherein said virus is chimeric.

3. The recombinant CMV virus of claim 2, wherein said virus is a Toledo-Towne chimera.

4. The recombinant CMV virus of claim 1, wherein said virus is selected from the group consisting of:
a. Toledo;
b. Towne;
c. AD169; and
d. Davis.

5. The recombinant CMV virus of claim 1, wherein said virus is attenuated.

6. The recombinant CMV virus of claim 1, wherein said heterologous polynucleotide is derived from a virus selected from the group consisting of:
a. HCV;
b. HIV;
c. RSV;
d. PIV;
e. HMPV; and
f. coronaviruses.

7. The recombinant CMV virus of claim 1, wherein the heterologous polynucleotide encodes a polypeptide selected from the group consisting of: HIV Env polypeptide, HIV Gag polypeptide, and HIV Pol polypeptide.

8. The recombinant CMV virus of claim 1, wherein said heterologous polynucleotide is derived from a pathogen selected from the group consisting of: M. tuberculosis; and Leishmania.

9. The recombinant CMV virus of claim 1, wherein said heterologous polynucleotide encodes at least one cancer antigen.

10. A recombinant CMV virus expressing a heterologous polynucleotide encoding at least one polypeptide, wherein said heterologous polynucleotide is operatively linked to a CMV pp65 promoter.

11. (canceled)

12. An immunogenic composition comprising the recombinant CMV virus of claim 1 and an excipient.

13. (canceled)

14. The recombinant CMV virus of claim 10, wherein said virus is attenuated.

15. The recombinant CMV virus of claim 10, wherein said heterologous polynucleotide is derived from a virus selected from the group consisting of:
a. HCV;
b. HIV;
c. RSV;
d. PIV;
e. hMPV; and
f. coronaviruses.

16. The recombinant CMV virus of claim 10, wherein said heterologous polynucleotide is derived from a pathogen selected from the group consisting of: M. tuberculosis; and Leishmania.

17. The recombinant CMV virus of claim 10, wherein said heterologous polynucleotide encodes at least one cancer antigen.

18. An immunogenic composition comprising the recombinant CMV virus of claim 10 and an excipient

19. A method of stimulating a cellular immune response comprising administering to a human the recombinant CMV virus of claim 1.

20. The method of claim 19, wherein the cellular immune response is a cytotoxic T lymphocyte (CTL) response.

21. A method of stimulating a cellular immune response comprising administering to a human the recombinant CMV virus of claim 10.

22. The method of claim 21, wherein the cellular immune response is a CTL response.


SEQUENCE LISTING (2 sequences)

SEQ ID NO: 1 - protein, 94 aa, Hepatitis C virus
IASPKGPVIQMYTNVDQDLVGWPAPQGSRSLTPCTCGSSDLYLVTRHADVIPVRRRGDSRGSLLSPRPISYLKGSSGGPLLCPAGHAVGLFRAA

SEQ ID NO: 2 - protein, 94 aa, Hepatitis C virus
IASPKGPVIQMYTNVDQALVGWPAPQGSRSLTPCTCGSSDLYLVTRHADVIPVRRRGDSRGSLLSPRPISYLKGSAGGPLLCPAGHAVGLFRAA